(19) United States
(12) Patent Application Publication
(10) Pub. No.: US 2024/0120095 A1
(43) Pub. Date: Apr. 11, 2024

(54) AI-ENABLED RISK ASSESSMENT OF ADVERSE HEALTH OUTCOME

(71) Applicant: HeartLung Corporation, Houston, TX (US)

(72) Inventors: MORTEZA NAGHAVI, HOUSTON, TX (US); CHENYU ZHANG, Los Angeles, CA (US); Kyle Atlas, Huntington Beach, CA (US)

(73) Assignee: HeartLung Corporation, Houston, TX (US)

(21) Appl. No.: 18/356,444

(22) Filed: Jul. 21, 2023

Related U.S. Application Data
(63) Continuation-in-part of application No. 18/167,852, filed on Feb. 11, 2023.
Application No. 18/167,852 is a continuation-in-part of application No. 18/167,691, filed on Feb. 10, 2023.
(60) Provisional application No. 63/414,546, filed on Oct. 9, 2022.
(60) Provisional application No. 63/414,561, filed on Oct. 9, 2022.

(51) Int. Cl. G16H 50/20; G16H 30/40

(52) U.S. Cl. CPC G16H 50/20; G16H 30/40

(57) ABSTRACT

Disclosed are systems and methods for determining an individual's risk of an adverse health outcome particularly a near-term cardiovascular event. In one embodiment, non-invasive chest CT scan images are input into an artificial intelligence system (AIS). Based on the CT scans, the AIS provides an analysis that includes one or more of a CAC score based; plaque density; plaque quantity; plaque locations; and the volume of cardiac chambers; and a measurement of cardiac ejection fraction (EF). Based on the AI analysis and additional risk factors, estimates of risk are determined. The estimation of risk can be done automatically by a digital application. In one embodiment, EF can be determined by the AIS using non-contrast, ECG-gated CT scan images acquired during end-diastole and end-systole. In some embodiments, the density of vasa vasorum around a coronary plaque is measured to detect active and inflamed plaques from passive and stable ones.

### RELATED APPLICATIONS

This application claims priority to U.S. patent applications 63/414,546 and 63/414,561, both filed on Oct. 9, 2022, and are hereby incorporated herein in their entireties by reference. This application is a continuation-in-part of co-pending U.S. patent application Ser. No. 18/167,691, filed on Feb. 10, 2023, which is hereby incorporated herein in its entirety by reference. This application is a continuation-in-part of co-pending U.S. patent application Ser. No. 18/167,852, filed on Feb. 11, 2023, which is a continuation-in-part of U.S. application Ser. No. 17/657,754, filed on Apr. 2, 2022, which are both hereby incorporated herein in their entireties by reference.

### BACKGROUND

### Field

The inventive subject matter is generally directed towards systems and methods for facilitating risk assessment of adverse health outcomes. In particular, embodiments of the invention relate to AI-based systems and methods for determining risks of adverse cardiovascular outcomes based, at least in part, on the use of a chest computerized tomography (CT) scan and AI-enabled analysis of the signals detected in the CT scan based on coronary artery calcifications (CAC) and cardiac chambers volumetry. Additional features include one or more of cardiac ejection fraction, pericardial fat, thoracic aortic calcification, aortic valve calcification, mitral valve calcification, pulmonary artery hypertension, emphysema, chronic obstructive pulmonary disease (COPD), fatty liver disease and bone mineral density.

### Description of Related Art

It is estimated that over 15 million individuals die every year from cardiovascular disease (CVD), with the majority being unaware of their risk until they experience a symptomatic life threatening condition which is often too late to reverse. Coronary Artery Disease (CAD) specifically and atherosclerotic cardiovascular disease (ASCVD) generally are the largest part of CVD death worldwide. Early detection of high risk ASCVD is crucial, particularly for asymptomatic individuals, as common symptoms such as chest pain and shortness of breath often do not manifest until a few minutes before a sudden cardiac death.

CAC scoring has been used to prove the presence of CAD and to facilitate predicting the risk of serious coronary events. A CAC score facilitates risk prediction and is more predictive than any other single biomarker. Adding a CAC score to traditional risk factors improves the Area Under the Curve (AUC) for ASCVD risk prediction.

The Agatston method quantifies the amount of calcium in coronary arteries. The Agatston score is a measure of coronary artery calcification obtained from a computed tomography (CT) scan of the heart. It is calculated by adding the scores of individual calcified plaques in the coronary arteries, considering the peak intensity of a calcified signal and area of each calcified plaque as defined by HU over 130. The resulting score represents the overall burden of calcium in the coronary arteries and serves as an indicator of an individual's CAD risk. A higher Agatston score indicates a greater amount of calcium buildup in the coronary arteries, correlating with a higher risk of ASCVD. The score can range from 0 (no detectable calcium) to several thousand, with scores above 400 indicating a high risk of coronary artery disease. The Agatston score is one of several methods used to assess a person's risk of heart disease, and is often combined with other factors, such as age, gender, and family history, to determine overall risk.

There is an unmet need for early detection of pre-symptomatic CVD, and for affordable, reliable, and scalable technology. While CAD can be assessed by a coronary artery calcium (CAC) scan which is a far more powerful predictor of ASCVD death than blood pressure and lipid levels, it is far from perfect. For example, in the NIH sponsored multi-ethnic study of atherosclerosis (MESA) over 15 years follow up of 6814 people, 99 people out of those with Agatston CAC=0 had an adverse coronary event.

Short-Term versus Long-Term Risk Prediction. CVD has been the primary cause of death and healthcare costs in the US for decades. Every year over 600,000 first-time heart attacks unexpectedly hit asymptomatic Americans. Currently less than 3% of US adults aged 20-79 years have an optimal cardiovascular risk factors profile defined as: total cholesterol <200 mg/dL (5.17 mmol/L), blood pressure <120/<80 mm Hg, non-smoker, body mass index (BMI)<25 kg/m2, fasting plasma glucose <100 mg/dL (5.56 mmol/L). Nonetheless, the awareness on CVD risk factors is above 95% meaning that almost all US adults are aware of the risk associated with these risk factors. Clearly, new strategies are needed.

Since the pioneering Framingham Heart Study in 1960s introduced CVD risk factors, preventive cardiology has focused on long-term CVD risk prediction. Currently, physicians tell their patients that based on their risk factors (age, gender, blood pressure, cholesterol, diabetes, smoking, etc.) their risk of developing CVD in the next 10 years is X. The median and mean for X are 2.7% and 5.2% respectively. Although such a long-term risk assessment is necessary, it is not enough. It does not trigger immediate preemptive actions and cannot detect asymptomatic patients who are vulnerable to a near-term CVD event. A layman's analogy to this scenario would be a TV weather broadcaster announcing that, over the next 10 years, a catastrophic hurricane will hit your area. Such an announcement would hardly change behaviors. However, when the weatherman displays a hurricane eye coming your way in the near future, it can cause immediate preemptive actions. A medical analogy would be finding a tumor in a cancer patient that gets serious attention and triggers immediate interventions to improve outcomes. Having a near-term predictive tool in cardiology might cause a paradigm shift resulting in developing new treatments. Because of the multi-factorial nature of CVD, the solution is preferably a multi-factorial prediction model based on several variables.

For CAD, screening and diagnostic tools like CAC score and coronary CT angiography are known. To predict atrial fibrillation (AF) and heart failure (HF), CHARGE-AF and brain natriuretic peptide (BNP) are used. CHARGE AF is an epidemiological risk calculator, not suitable for an individualized risk assessment and monitoring, while BNP is more precise but not specific to, for example, left atrial and ventricular function.

AF is the most common sustained arrhythmia and carries an increased risk of stroke and cardiovascular mortality. In the United States, at least 3 to 6 million people experience AF. The number of AF patients is expected to rise to over 12 million cases by 2030, imposing a significant economic burden of $260 million in healthcare expenses. In Europe, AF among those older than 55 years is projected to reach 14 million by 2040. It is estimated that by 2050, at least 72 million people in Asia will be diagnosed with AF, and about 3 million with AF-related strokes. This presents a public health crisis especially for the growing elderly population in the coming decades.

Medicare services costs are significantly higher among AF patients than non-AF patients; therefore, early treatment is critical to limit the disease burden imposed by AF. The adverse social and public health effects of HF are even worse. It is estimated that by 2030, more than 8 million people in the United States will have HF. And the total direct medical costs of HF are expected to rise from $21 billion to $53 billion.

The 5-year survival rates of AF are of concern. Without proper treatment, 51% of AF patients will die within five years. Although the economic burden posed by AF and HF are critical considering the increasing healthcare costs, early detection tools and preventive interventions for pre-AF and pre-HF patients are currently unavailable. One report shows that 96,860 strokes occurred within 1 year among patients with AF, with an associated total direct lifetime cost of nearly $8 billion. Of these costs, $2.6 billion in direct costs are incurred during the first year after the stroke.

HF poses an even greater threat to the American healthcare system. Given the rising rates of hospitalization and re-hospitalization, HF is associated with a significant cost burden. Approximately 1% to 2% of the total US health care budget is spent on HF, and half of that is attributable to late diagnosis leading to inpatient admissions for HF. This challenge presents a great opportunity to make an impact on the healthcare system by early detection and interventions of subclinical HF and AF. Currently, BNP and CHARGE-AF are the only available tools for early detection of high-risk patients for AF and HF. A combination of high-risk CHARGE-AF and a 7-day ECG patch has been reported. CHARGE-AF is an epidemiological risk calculator that can be useful as a population-based measure, but it is not preferred as applied to individual patients as needed in a physician's office. A more direct assessment is preferred such as by imaging the cardiac chambers where AF happens.

It is known to use manual measurements of left ventricle chamber in a single slice to predict HF. However, rapid and accurate acquisition of whole heart volume parameters is challenging. Even though semi-automated delineation and quantification of cardiovascular structures can be useful in CT images, currently known methods still require a significant degree of manual modifications, which is time-consuming and may increase inter-/intra-observer variability.

Approximately half of all adults aged 50 and older are at risk of bone fractures due to osteoporosis or osteopenia. From 1990 to 2019, global deaths and disability-adjusted life-years (DALYs) attributable to low bone mineral density (BMD) increased by 112% and 94%, respectively. Proper treatment can prevent about half of the repeat fractures related to osteoporosis. Likewise, most osteopenia cases can be prevented and reversed with appropriate healthcare. However, a significant number of individuals with osteopenia and osteoporosis remain unaware of their bone loss. A BMD test serves as the sole method for early determination of a suitable treatment plan to prevent further bone loss and future fractures.

Dual-energy x-ray absorptiometry (DEXA) can be used for assessing BMD, but less than 20% of the population who should get BMD test undergoes one. DEXA is limited by its 2D planar technique making it unable to distinguish between cortical and trabecular bone, leading to underestimated bone loss, especially in overweight individuals. Further, osteoporosis is usually asymptomatic prior to the fracture event resulting in fractures becoming the dominant clinical manifestation. Consequently, osteoporosis remains an underdiagnosed and undertreated condition associated with high morbidity and mortality.

Lung cancer is also a major disease affecting humans. It is the second most common diagnosed cancer in the United States, and it accounts for the greatest number of cancer deaths in both men and women worldwide. Early detection of lung cancer has been challenging and it was not until 2011 with the release of data from the National Lung Cancer Screening Trial (NLST) that a screening test for lung cancer was demonstrated to reduce lung-cancer specific mortality. More specifically, this trial demonstrated that use of low dose computed tomography (LDCT) for lung cancer screening resulted in a significant reduction rate in lung cancer mortality, so that LDCT is now the standard of care for lung cancer screening.

Currently, no screening tool is available for detecting individuals at high risk of AF and/or HF. Despite the critical need no biomarker is currently available to identify individuals at high risk for AF or HF and their complications. An ideal biomarker would help to detect individuals at high risk for both AF and HF. In view of the above, there is a long-felt need in the healthcare industry to improve predictive risk assessment of adverse health outcomes in health care settings. Embodiments of the systems and methods disclosed here address these and other needs in the relevant art.

### SUMMARY OF ILLUSTRATIVE EMBODIMENTS

Data integration projects face challenges bringing together the right expertise. There are pipeline behaviors that can be common across multiple pipelines. However, under known methods, typically pipeline behaviors are not developed once and made reusable by different pipelines. Some embodiments disclosed herein address the need for scale where dozens of projects may be concurrently active.

In one aspect, the invention is directed to an AI-based method of assessing the risk of adverse health outcomes. In one embodiment, the method involves providing an artificial intelligence system (AIS) configured to analyze a set of computed tomography (CT) scan images; inputting a set of CT scan images into the AIS for analysis; receiving an AI analysis from the AIS, the AI analysis including: (a) a coronary artery calcium (CAC) score based, at least in part, on a modified Agatston score; (b) a measurement of mean Hounsfield units (HU) per plaque adjusted by plaque volume; (c) a measurement of plaque quantity per coronary artery and per patient; (d) a measurement of the number of coronary arteries with one or more plaque; (e) a measurement of location of plaques from proximal, to aortic root, to distal coronary artery; (f) a volume of at least one cardiac chamber; wherein the AI analysis is based at least in part on the set of CT scan images; and determining, based at least in part on a computerized risk calculator, the AI analysis, and known risk factors, a patient's risk for an adverse health outcome.

In some embodiments, the known risk factors include one or more of age, gender, ethnicity, smoking, blood pressure, blood lipids, blood glucose, hemoglobin A1C, brain natriuretic peptide, presence of diabetes, endothelial dysfunction, and cardiometabolic syndromes. In certain embodiments, the AI analysis further includes a measurement of at least one of: plaque shape, distance from other plaques, distribution of plaques mean HU per coronary artery, aortic valve calcification, aortic wall calcification, aortic diameter, pulmonary arteries diameter, left atrium, left ventricle, right atrium, right ventricle, thoracic bone mineral density, pericardial fat, and liver fat, intra thoracic fat, emphysema, lung nodules, subcutaneous fat, and thoracic muscle mass.

In certain embodiments, determining a patient's risk for an adverse health outcome further comprises configuring the risk calculator for specialized assessment of different adverse outcomes selected from the group including coronary heart disease, congestive heart failure, atrial fibrillation, left ventricular hypertrophy, stroke, chronic obstructive pulmonary disease, cardiovascular death, and all-cause mortality. In one embodiment, determining a patient's risk for an adverse health outcome further comprises configuring the risk calculator to facilitate specialized short-term risk assessment of adverse health outcomes to provide an alert of an imminent risk, wherein the short-term can be days, weeks, and/or up to 12 months.

In some embodiments, the AI analysis further includes a measurement of plaque calcifications defined by HU≥100. In certain embodiments, the set of CT scan images includes images obtained from contrast-enhanced cardiac CT scans. In one embodiment, the method can further include monitoring the amount of calcification in the coronary arteries, cardiac and aortic valve areas overtime to track progression or regression of aortic valve calcification and gauging response to treatments. In some embodiments, the AI analysis can further include a display of coronary arteries vasa vasorum density around coronary arteries by mapping the Hounsfield units where the areas corresponding to highest vasa vasorum density have the highest Hounsfield units.

In another aspect the invention concerns an AI-based system for facilitating risk assessment of adverse health outcomes. In one embodiment, the system can include a device configured to facilitate acquiring and storing a set of CT scan images; and an AI-enabled analyzer configured to generate an analysis based, at least in part, on said set of scan images. The analysis preferably includes a coronary artery calcium (CAC) score based, at least in part, on an Agatston score; a measurement of plaque density; a measurement of plaque quantity; one or more locations of plaque; and a volume of at least one cardiac structure. The system can further include a computer-enabled risk calculator configured to determine, based at least in part on the analysis, a particular patient's risk for an adverse health outcome.

In some embodiments, the analysis can further include a measurement of thoracic aortic calcification and a measurement of bone mineral density. In certain embodiments, the computer-enabled risk calculator is further configured to determine the patient's risk for an adverse health outcome based, at least in part, on at least one known risk factor selected from the group: age, gender, ethnicity, smoking, blood pressure, blood lipids, blood glucose, hemoglobin A1C, brain natriuretic peptide, presence of diabetes, endothelial dysfunction, and cardiometabolic syndromes.

In certain embodiments, the computer-enabled risk calculator is configured to determine a particular patient's risk for an adverse health outcome selected from: coronary heart disease, congestive heart failure, atrial fibrillation, stroke, chronic obstructive pulmonary disease, cardiovascular death, and all-cause mortality. In yet other embodiments, the computer-enabled risk calculator is further configured to facilitate specialized short-term risk assessment of adverse health outcomes to provide an alert of an imminent risk; the short-term can be days, weeks, and/or up to 12 months. In certain embodiments, the AI analysis further includes a measurement of plaque calcifications defined by HU≥100.

Yet another aspect of the invention relates to an AI-enabled method of measuring cardiac ejection fraction (EF). In one embodiment, the method involves providing an artificial intelligence system (AIS) configured to estimate cardiac chambers volumes based on non-contrast ECG-gated CT scan images of the heart; providing a first image comprising a non-contrast, ECG-gated CT scan image of the heart acquired during end-diastolic period; providing a second image comprising a non-contrast, ECG-gated CT scan image of the heart acquired during end-systolic period; wherein the first and second measurements are based, at least in part, on the first and second images; and calculating the difference in left ventricular volume between the first and second images; and determining an EF measurement based, at least in part, on the first and second measurements.

In some embodiments, the first image is acquired during isovolumetric contraction, and wherein the second image is acquired during isovolumetric relaxation. In certain embodiments, the method can further include receiving from the AIS a third measurement comprising a measurement of LV wall volume and total heart volume to calculate total heart volume changes between end-diastolic period and end-systolic period.

Another aspect of the invention is directed to an AI-enabled system (AIS) configured to facilitate detecting individuals at high risk of future adverse health outcomes. In one embodiment, the system includes an AI-based module configured to receive and analyze 2-D chest X-ray images; and a computerized calculator configured to detect individuals at high risk of one or more of: atrial fibrillation, heart failure, and stroke; wherein the AI-based module is configured to perform an analysis based, at least in part, on detecting in the X-ray images characteristics of an enlarged left atrium, enlarged left atrial appendage, enlarged right atrium, dilated pulmonary arteries, dilated pulmonary veins, and/or enlarged right and left ventricles.

Yet another aspect of the invention concerns an AI-enabled system (AIS) configured to improve the clinical utility of coronary artery calcium (CAC) scans. In one embodiment, the system includes an AI-based module configured to extract from the CAC scans cardiac chambers volumetry data and an Agatston CAC score; and a computerized CVD risk calculator configured to provide a minimum Net Reclassification Index (NRI) of over 0.1 for risk assessment of individuals at risk of future heart failure, atrial fibrillation, stroke, CVD related death, and all-cause mortality; and wherein the computerized CVD risk calculator is configured to provide the NRI based, at least in part, on the cardiac chambers volumetry data and the Agatston CAC score.

Another aspect of the invention is directed to an AI-based method of assessing the risk of adverse health outcomes. In one embodiment, the method includes providing a first artificial intelligence system (AIS1) configured to analyze a set of computed tomography (CT) scan images; inputting a set of CT scan images into AIS1 for analysis; receiving an AI analysis from AIS1, the AI analysis including: a coronary artery calcium (CAC) score based, at least in part, on an Agatston score; a measurement of mean, median, standard deviation, and range of Hounsfield units (HU) per plaque adjusted by plaque volume; a measurement of plaque quantity per coronary artery and per patient; a measurement of the number of coronary arteries with one or more plaque; a measurement of location of plaques from proximal to the aortic root, to distal parts of a coronary artery; a volume of at least one cardiac chamber; wherein the AI analysis is based at least in part on the set of CT scan images; providing a second AIS (AIS2) configured to classify data associated with coronary plaques and/or cardiac chambers based on the output of AIS1; and determining, based at least in part on a computerized risk calculator, the AIS1 analysis, the classification performed by AIS2, and known risk factors, a patient's risk for an adverse health outcome.

One more aspect of the invention concerns an AI-based method of assessing and monitoring changes in the risk for adverse health outcomes. In one embodiment, the method includes providing an artificial intelligence system (AIS) configured to analyze a set of computed tomography (CT) scan images and to output an analysis comprising a cardiovascular risk score based on five or more measurement from the group comprising: a measurement of the Agatston calcium score; a measurement of mean, median, standard deviation, and range of Hounsfield units (HU) per plaque adjusted by plaque volume; a measurement of plaque quantity per coronary artery and per patient; a measurement of the number of coronary arteries with one or more plaque; a measurement of location of plaques from proximal to the aortic root, to distal parts of a coronary artery; a measurement of the length of the longest axis of a plaque; a measurement of proximity of plaques to each other within a coronary artery; a volume of one or more cardiac chambers comprising left atrium, left ventricle, right atrium, right ventricle, and left ventricular wall; a measurement of bone mineral density using average HU in trabecular bones; a measurement of lung nodules; and a measurement of lung emphysema score. In some embodiments, the AI analysis output can be inputted into a computerized risk calculator configured to determine, based on AIS analysis output and one or more of known risk factors, a patient's risk for an adverse health outcome.

Additional features and advantages of the embodiments disclosed herein will be set forth in the detailed description that follows, and in part will be clear to those skilled in the art from that description or recognized by practicing the embodiments described herein, including the detailed description which follows, the claims, as well as the appended drawings.

Both the foregoing general description and the following detailed description present embodiments intended to provide an overview or framework for understanding the nature and character of the embodiments disclosed herein. The accompanying drawings are included to provide further understanding and are incorporated into and constitute a part of this specification. The drawings illustrate various embodiments of the disclosure, and together with the description explain the principles and operations thereof.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 is a flowchart of a method of facilitating assessing a risk of an adverse health outcome according to one embodiment of the invention.

FIG. 2 is a block diagram of a system for facilitating assessing a risk of an adverse health outcome according to one embodiment of the invention.

FIG. 3 is a flowchart of another method of facilitating assessing a risk of an adverse health outcome according to one embodiment of the invention.

FIG. 4 is a graph showing cumulative incidence of HF by LV volume quartiles as performed by certain embodiments of the systems and methods disclosed herein.

FIG. 5 is a graph showing a density plot of HErEF and HEpEF by LV volume index as performed by a prior art method.

FIG. 6 is a graph showing a density plot HErEF and HEpEF by LV volume index as performed by certain embodiments of the systems and methods disclosed herein.

FIG. 7 is a graph showing a prediction model performance, as performed by the inventive methods and system disclosed herein, against a prediction model performed by conventional methods.

FIG. 8-10 are graphs illustrating computer-enabled risk calculators for, respectively, 1-year, 2-year, and 3-year follow-up prediction of stroke, according to certain embodiments of the invention disclosed here.

FIG. 11-13 are graphs illustrating computer-enabled risk calculators for, respectively, 1-year, 2-year, and 3-year follow-up prediction of atrial fibrillation (AF), according to certain embodiments of the invention disclosed here.

FIG. 14-16 are graphs illustrating computer-enabled risk calculators for, respectively, 1-year, 2-year, and 3-year follow-up prediction of congestive heart failure (CHF), according to certain embodiments of the invention disclosed here.

FIG. 17 is a graph of cumulative incidence of all coronary heart disease (CHD) based on a prior art Agatston CAC score.

FIG. 18 is a graph of cumulative incidence of all coronary heart disease (CHD) based on a modified Agatston CAC score (Agatston+) according to some embodiments of the invention disclosed here.

FIG. 19 is an example of a 2D image (an X-ray scan) analyzed for use with certain embodiments of the invention disclosed here.

FIG. 20 illustrates a method, according to certain embodiments of the invention, of determining an adverse health risk of a patient.

### DETAILED DESCRIPTION OF ILLUSTRATIVE EMBODIMENTS

The specific details of the single embodiment or variety of embodiments described herein are set forth in this application. Any specific details of the embodiments are used for demonstration purposes only, and no unnecessary limitation or inferences are to be understood therefrom.

Before describing in detail exemplary embodiments, it is noted that the embodiments reside primarily in combinations of components related to the system. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

HeartLung Corporation makes available several AI-enabled tools: coronary artery calcium score (AutoCAC) using non-contrast chest CT scans; bone mineral density (AutoBMD) measurements; and cardiac structure volumetry (AutoChamber). AutoCAC can also provide density, quantity, and location of plaques. AutoChamber can also provide information associated with pericardial fat, thoracic aorta calcium, aorta, and pulmonary aorta sizing. HeartLung's technology can work on new scans and existing scans.

There are imaging features of coronary calcification that can enable improving the risk stratification particularly in patients with calcium score of 1 to 100. These features include calcium density (scattered versus dense), shape of calcification (sharp versus blunt edges), heterogeneity (focal or segmental around the calcified spot) and distribution (distal and proximal location). Calcium density is not properly used in Agatston scoring. The Agatston score is weighted based on the maximum intensity Hounsfield unit (HU) of pixels corresponding to a calcified plaque in the coronary arteries, but it does not take into account the average intensity (HU) of the calcified plaque neither it considers the distribution and standard deviation of HU. For a given plaque area, the Agatston score is increased 2, 3, or 4-fold based on reaching higher calcium density HU cut points which elevates the calcium score. However, densely calcified coronary plaques may pose a lower CVD risk than less densely calcified plaques because they are presumably more stable and have less lipid core. The importance of differences in the degree of atherosclerotic plaque calcification is studied in randomized trials with CAC and statin therapy. Older and more established plaques are more likely to calcify. Conversely, newer plaques have less calcium, greater lipid content and are more likely to be vulnerable. Deep learning techniques can facilitate identification of specific imaging features that can differentiate individuals and reclassify those at risk of having cardiovascular events with low calcium score (1-100) or zero calcium score.

One of the embodiments of this inventive matter teaches a significant improvement over Agatston Score and is here referred to as the modified Agatston method. Unlike the Agatston score, in one embodiment, the modified Agatston method is weighted by the number of plaques, mean HU intensity (not maximum HU intensity that Agatston uses) adjusted by number of pixels, location of plaque, and/or vessels. An Agatston score 1-100 is a weakness of coronary artery calcium scoring using the prior art Agatston method. This is similar to the weakness of the Intermediate Risk category of existing ASCVD risk factors-based scoring method (ASCVD score), which is widely known to be inaccurate in the Intermediate Risk category where most heart attacks happen instead of in the High Risk category. In the case of the existing Agatston Score, about 25% of adverse events happen in the 1-100 Agatston score category. The modified Agatston facilitates identifying individuals who are actually High Risk in this CAC 1-100 population and should not take a CAC 1-100 lightly. This is a major improvement over the existing Agatston CAC score.

In addition to enhanced CAC features, certain embodiments of the invention disclosed here measure quantitative radiomic features of other components in a chest CT scan including cardiac chambers volume, pericardial fat volume, aorta and pulmonary trunk diameters, liver density, aortic calcification, aortic valve calcification, and/or mitral valve calcium.

Using the Agatston score, two individuals can have a CAC 200 score. The individual with a higher number of plaques, or with the lower density of plaques (lower HU), is at higher risk. If there is only one plaque, the individual with the plaque located proximally close to the origin of coronaries is at higher risk than the individual with the plaque located mid or distal. Therefore, the usefulness of a CAC score can be increased if additional information regarding the number, density, distribution, and location of plaques is obtained.

A chest CT scan can be a suitable tool for evaluating the risk of an individual for two leading causes of mortality and morbidity, namely CVD and lung cancer. A chest CT scan can provide information about both the cardiovascular system and the pulmonary system. Applying artificial intelligence (AI) to chest CT scans can empower a provider to rapidly extract actionable information for evaluating the risk of adverse health events not only related to CVD and lungs (that is, detecting pre-cancerous lung nodules, silent emphysema, coronary calcification, aortic calcification, cardiac valves calcification, cardiac chambers enlargement, aortic enlargement, pulmonary artery enlargement, etc.), but it can also detect osteoporosis and osteopenia by measuring thoracic vertebral bone density, cardiometabolic associated excess intrathoracic visceral fat, fatty liver disease, muscle loss or sarcopenia, and other abnormalities in other organs such as thyroid nodules and esophageal masses. All these findings can be actionable and can be automatically detected by a suitably trained artificial intelligence system in both non-contrast and contrast enhanced chest CT scans.

Artificial intelligence (AI) can be used in medical imaging, and AI performance can be fine-tuned to facilitate detection and quantification of various clinical conditions. BMD measurement often requires additional manual work. The systems and methods disclosed here can be a major add-on to CAC and lung cancer scans. The methods and systems disclosed here can be advantageous in identifying individuals at risk of adverse health outcomes even when the individuals are asymptomatic.

CAC score and lung cancer screening CT scans are obtained each year in large quantities. CT-based assessment of BMD measurement for osteoporosis screening, initially manually and then with automated approaches. A fully automated Deep Learning (DL) model has the potential of increasing the reach of CT-based osteoporosis screening.

AI-based detection can measure BMD for both full chest and cardiac CT scans with an accuracy level comparable to manual measurements. For the same patient, there are comparable results obtained between three of the vertebral bones found in cardiac and lung CT scans, respectively. BMD assessment can be carried out with either a cardiac or a lung CT scan and provide the same results clinically for the same patient. In one embodiment, a BMD measurement tool can offer unique and significant advantages, being that patients undergo no additional radiation nor additional trips to imaging centers and there is no additional scanner cost or additional scanning time.

BMD can be calculated from normal bone tissue of trabecular bone in specific spines. Thus, it is important to detect individual vertebrae and identify disk locations for removal from calculation.

Two DL models were developed for cardiac and full chest CT scan images, respectively. The models were trained to detect trabecular bone and disks with a training data set of 132 cardiac and 37 lung CT images. For ground truth, 225 cardiac and lung CT images for whole spine and disk locations were manually segmented. Each DL model has two steps to automatically detect individual vertebrae and disks. In the first step, the models were trained to focus on the whole spine area and trained for 100 epochs. Transfer learning was then used to train for disk locations using the pretrained models. In one embodiment, the architecture of the models can include an encoder and a decoder. The encoder can be a UNet with 12 layers of 2D convolutions, skip connections, Leaky ReLu activations and/or batch normalization. The decoder can be a 3-layer convolution 2D with Leaky ReLu activations and/or a sigmoid at the end.

After identifying individual vertebrae, a set of rules can be used to find the disk location. Vertebrae smaller or bigger than a standard size were removed from calculations. Signal processing was used to erode the borders to segment the entire vertebral bone.

BMD is measured in the trabecular bone in the center of each vertebra. To measure the BMD, the center of mass for each vertebra can be found. A cylinder with a radius of 1-ccm can be created where the center of the mass can be identified. The height of cylinder can be determined by taking two slices above and below adjacent disk locations to avoid cortical bones. The mean HU can be calculated from this volume.

A minimum of three individual vertebrae HU values can be calculated this way. In one embodiment, a case is dismissed if three separate measurements are not found. For each measurement a normalization factor for scanner type can be used to calculate BMD value. Using three vertebrae, T and Z values are calculated considering gender and age of the patient. The input to the model can be cardiac gated CT scans and the output can be, for example, a JSON file that includes the mean HUs, BMD, T score and Z score values. Two snapshots of sagittal and coronal views with superimposed spine and disk segmentations can also be rendered for quality control.

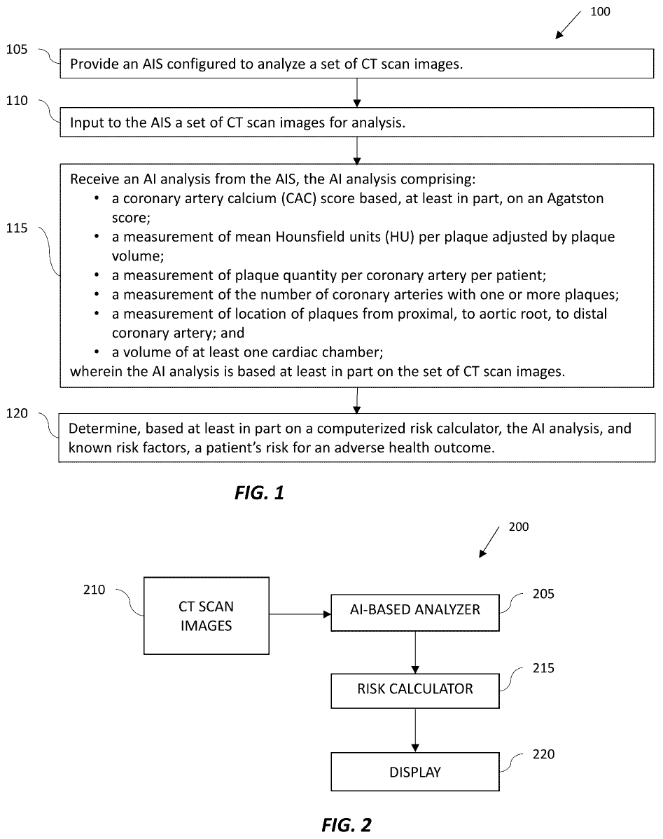

Referencing FIG. 1, method 100 of determining a patient's risk for an adverse health outcome is illustrated. In one embodiment, at step 105 an artificial intelligence system (AIS) is provided. The AIS can be an AI component that has been trained and configured to analyze a set of CT scan images. Such AIS can be, for example, AutoCAC, AutoBMD, and/or AutoChamber by HeartLung Corporation. At step 110, a set of non-contrast CT scan images is input to the AIS for analysis. In one embodiment, the set of non-contrast CT scan images is retrieved from, for example, a cloud storage system. In some embodiments, the set of non-contrast CT scan images is acquired during a patient's clinical visit, and the set of non-contrast CT scan images can be stored on a storage system local to the clinical setting. In certain embodiments, the AIS system can be configured to receive instructions to retrieve the set of non-contrast CT scan images from a storage system. In certain embodiments, the set of non-contrast CT scan images can include chest CT scan images and/or lung CT scan images.

At step 115, in one embodiment, an AI analysis from the AIS is received. In certain embodiments, the AI analysis can include, for example, a coronary artery calcium score based, at least in part, on an Agatston score; a measurement of plaque density; a measurement of plaque quantity; one or more locations of plaque; a volume of at least one cardiac structure; and a measurement of cardiac ejection fraction (EF). In one embodiment, the AI analysis is based at least in part on the set of non-contrast CT scan images. In some embodiment, the AI analysis can include one or more measurements of: pericardial fat, thoracic aortic calcification, and bone mineral density.

At step 120, based at least in part on the AI analysis, a patient's risk for an adverse health outcome is determined. In one embodiment, at least one estimate of risk for an adverse health outcome is determined, at least in part, by a computer-enabled risk calculator. For example, based at least on a volume of at least one cardiac structure, a computer-enabled risk calculator can compute a patient's risk for heart failure. By way of another example, based at least on EF, a computer-enabled risk calculator can compute a risk of future heart failure, myocardial infarction, and/or sudden cardiac death. In certain embodiments, the adverse health outcome can be osteopenia and/or osteoporosis.

FIG. 2 illustrates system 200 for facilitating determining a risk of an individual for an adverse health condition according to one embodiment of the invention disclosed here. In one embodiment, system 200 can include AI-based analyzer 205. In some embodiments, AI-based analyzer 205 includes an AI model configured to facilitate determining one or more measurements and/or scores associated with cardiovascular health. In certain embodiments, AI-based analyzer 205 can be configured to produce an AI-based analysis including one or more of a coronary artery calcium score based, at least in part, on an Agatston score; a measurement of plaque density; a measurement of plaque quantity; identification of one or more locations of plaque; a volume of at least one cardiac structure; a measurement of cardiac ejection fraction (EF); pericardial fat; thoracic aortic calcification; and bone mineral density. In one embodiment, AI-based analyzer 205 can include, for example, one or more of AutoCAC, AutoBMD, and/or AutoChamber by HeartLung Corporation. In some embodiments, AI-based analyzer 205 can include a first artificial intelligence system (AIS1) configured to read and/or analyze chest CT scans. AI-based analyzer 205 can further include a second artificial intelligence system (AIS2) configured to classify, based on the output of AIS1, high-risk plaque versus low-risk plaque.

In certain embodiments, system 200 can include CT scan images 210, which can be stored in a computer memory, for example. CT scan images can include images obtained from CT scans, for example, contrast enhanced CT scans, non-contrast enhanced CT scans, ECG-gated cardiac CT scans, non-gated cardiac CT scans, non-gated full chest CT scans, low dose lung cancer screening CT scans, and a combination of contrast enhanced and non-contrast enhanced chest CT scans. In some embodiments the cardiovascular structure can be, for example, left atrium (LA), left ventricle (LV), left ventricular wall (LVW), right atrium (RA), right ventricle (RV), aorta, and/or pulmonary artery.

System 200 can, in one embodiment, include risk calculator 215 configured to take as input one or more of the measurements or scores from the AI-based analysis to determine, based at least in part on the measurements or scores, a risk of a patient for an adverse health condition. In certain embodiments, the adverse health condition can be, for example, atrial fibrillation (AF), heart failure (HF), stroke, cerebrovascular events, chronic obstructive pulmonary diseases (COPD), emphysema, ischemic heart disease, cardiovascular mortality, and all-cause mortality. In some embodiments, the risk is determined based on the estimated measurement and/or score and considering other variables, such as patient's age, gender, height, weight, body surface area, body mass index, and ethnicity, for example. In some embodiments, the estimated volume can be used with one or more health related variables to enhance the prediction model, resulting in a multivariate composite index of health to better determine the risk of future adverse health conditions. In certain embodiments, the one or more health related variables can be, for example, blood pressure, heart rate, blood oxygenation, blood tests, medications, and other patient medical data. In some embodiments, AI-based analyzer 205 and risk calculator 215 can be implemented as a single component that, for example, receives CT scan images 210 as input and returns risk estimates as output shown in display 220. In certain embodiments, risk calculator 215 can be a comprehensive risk assessment module configured to combine the output of AIS2 with, for example, known emerging risk factors. Said risk assessment module can be, for example, an AIS classifier or a multi-variate model that takes into account CT scan analysis and non-image data.

Polygenic risk score (PRS) is a quantitative measure that combines information from numerous genetic markers across an individual's genome to estimate their genetic predisposition for developing CVD. In some embodiments, risk calculator 215 can use PRS to provide valuable insights into an individual's susceptibility to CVD, thereby enabling early identification of high-risk individuals based on a comprehensive set of data that includes genetic susceptibility information, imaging biomarkers, epidemiological risk factors, circulating biomarkers, and physiological information. Use of PRS in system 200 facilitates improving risk assessment for early preventive strategies, and personalized treatment approaches for CVD. PRS is primarily a long-term indicator of risk; however, environment-genes interactions make the role of PRS a lot more complex. In certain embodiments of system 200, an AIS can be configured to stratify risk based on the weight of each component per individual. For example, the role of PRS in an 80-year-old male can be assigned less weight than the physiological findings or imaging information from the heart. In contrast, in a 40-year-old male the PRS can be assigned a higher weight and, consequently, plays a greater role in developing a personalized risk reduction and preventive plans.

In one embodiment, risk calculator 215 can include a software module configured to determine, for example, the ten-year risk of Hard CVD outcome for a female individual by implementing the formula: 10 yr risk=1−(0.9757\hat{ }exp(0.0635*age −0.0190*HDL+0.4353*(HTN)+0.8623*Smoker+0.0400*LAVI+0.0518*LVVI −0.0566*RVVI −3.3674)). Similar equations can be derived and implemented based on exemplary values provided in the tables below. It should be noted that not all variables need be included in a given risk calculator formula.

Here, age is the individual's age, total cholesterol is the individual's total cholesterol measurement, HDL is the individual's HDL measurement, HTN is presence of hypertension, smoker is a binary variable indicating status as smoker or non-smoker, LAVI is the individual's left atrium volume index, LVVI is the individual's left ventricle volume index, and RVVI is the individual's right ventricle volume index. In some embodiments, LAVI, LVVI, and RVVI can be determined by dividing the cardiac chamber volume by the individual's weight. This results in normalizing the cardiac chamber volume and making it comparable across individuals.

| | | | |
|---|---|---|---|
| 10-YR | Hard CVD | All CVD | Heart Failure |

| RISK | F | M | F | M | F | M |
|---|---|---|---|---|---|---|
| age | 0.0635 | 0.0512 | 0.0566 | 0.0519 | 0.0478 | 0.0712 |
| Total chol | | | | 0.0048 | | |
| HDL | −0.0190 | −0.0095 | −0.0153 | −0.0112 | | |
| SBP | | | | 0.0084 | | |
| HTN | 0.4353 | | 0.4805 | | | |
| Smoker | 0.8623 | 0.5809 | 0.7007 | 0.4889 | 0.6687 | |
| Diabetes | | 0.6733 | | 0.7073 | 1.1845 | 0.7641 |
| LAVI | 0.0400 | | 0.0357 | | 0.0486 | |
| LVVI | 0.0518 | | 0.0389 | | 0.0921 | |
| RAVI | | | | | | |
| RVVI | −0.0566 | −0.0341 | −0.0509 | −0.0221 | −0.0824 | −0.0466 |
| LVWI | | 0.0583 | | 0.0445 | | 0.1022 |
| Baseline | 0.9757 | 0.9461 | 0.9638 | 0.9192 | 0.9883 | 0.9757 |
| survival | | | | | | |
| Mean | 3.3674 | 3.8626 | 2.7332 | 5.8747 | 4.0382 | 7.1913 |
| (var) × coef | | | | | | |

| | | |
|---|---|---|
| 10-YR | AFib | Stroke |

| RISK | F | M | F | M |
|---|---|---|---|---|
| age | 0.0635 | 0.0512 | 0.0566 | 0.0519 |
| Total chol | | | | 0.0048 |
| HDL | −0.0190 | −0.0095 | −0.0153 | −0.0112 |
| SBP | | | | 0.0084 |
| HTN | 0.4353 | | 0.4805 | |
| Smoker | 0.8623 | 0.5809 | 0.7007 | 0.4889 |
| Diabetes | | 0.6733 | | 0.7073 |
| LAVI | 0.0400 | | 0.0357 | |
| LVVI | 0.0518 | | 0.0389 | |
| RAVI | | | | |
| RVVI | −0.0566 | −0.0341 | −0.0509 | −0.0221 |
| LVWI | | 0.0583 | | 0.0445 |
| Baseline | 0.9757 | 0.9461 | 0.9638 | 0.9192 |
| survival | | | | |
| Mean | 3.3674 | 3.8626 | 2.7332 | 5.8747 |
| (var) × coef | | | | |

| 10-YR Risk | | |
|---|---|---|
| for HF | Females | Males |
| age | 0.0205 | 0.0587 |
| Total chol | | |
| HDL | | |
| SBP | | |
| HTN | | |
| Smoker | | |
| Diabetes | 1.0783 | 0.7107 |
| LAVI | 0.0438 | |
| LVVI | 0.0900 | |
| RAVI | | |
| RVVI | −0.0773 | −0.0445 |
| LVWI | | 0.1022 |
| Ln(Agatston + 1) | 0.1817 | 0.1113 |
| Baseline survival | 0.9860 | 0.9746 |
| Mean (var) × coef | 2.6251 | 6.8936 |
| 10-YR Risk | | |
| for Stroke | Females | Males |
| age | 0.0051 | 0.0159 |
| Total chol | | |
| HDL | −0.0212 | |
| SBP | 0.0111 | 0.0168 |
| HTN | | |
| Smoker | 0.6877 | |
| Diabetes | | 0.7111 |
| LAVI | 0.0389 | 0.0722 |
| LVVI | 0.0459 | |
| RAVI | | −0.0597 |
| RVVI | −0.0495 | |
| LVWI | | |
| Ln(Agatston + 1) | | 0.1171 |
| Baseline survival | 0.9835 | 0.9806 |
| Mean (var) × coef | 4.0805 | 3.3928 |

| | 1-YR | | 2.5-YR | | 5-YR | | 10-YR | |
|---|---|---|---|---|---|---|---|---|
| AF Risk | Fem | Males | Fem | Males | Fem | Males | Fem | Males |
| age | 0.0829 | 0.0994 | 0.1084 | 0.1034 | 0.0869 | 0.0796 | 0.0797 | 0.0828 |
| height | | | | | | | | |
| weight | 0.0418 | 0.0634 | 0.0252 | 0.0217 | 0.0209 | 0.0132 | 0.0141 | 0.0102 |
| HDL | | | | | | | | |
| SBP | | | | | | | | |
| HTN | | | | | | | | |
| Smoker | | | | | | | | 0.3612 |
| Diabetes | | | | | | | | |
| LAVI | 0.0775 | 0.0993 | 0.0605 | 0.0691 | 0.0587 | 0.0606 | 0.0584 | 0.0413 |
| LVVI | | | | | | | | 0.0199 |
| RAVI | | | | | | | | |
| RVVI | | | | | | | | −0.0139 |
| LVWI | | | | | | | | |
| Ln(Agat + 1) | | | | | | | | 0.0771 |
| Baseline | 0.9978 | 0.9980 | 0.9938 | 0.9916 | 0.9802 | 0.9721 | 0.9504 | 0.9269 |
| Mean | 10.6336 | 14.8153 | 10.4643 | 10.5513 | 8.7620 | 8.0714 | 7.8004 | 7.7252 |

| 10-YR Risk | Females | | | Males | | |
|---|---|---|---|---|---|---|
| for All CVD | Univar | Full | Sign | Univar | Full | Sign |
| age | 0.0739 | 0.0272 | 0.0319 | 0.0537 | 0.0271 | 0.0293 |
| Total chol | 0.0011 | 0.0019 | | 0.0016 | 0.0041 | 0.0044 |
| HDL | −0.0138 | −0.0104 | −0.0131 | −0.0087 | −0.0106 | −0.0136 |
| SBP | 0.0215 | 0.0049 | | 0.0221 | 0.0067 | 0.0095 |
| HTN | 0.9152 | 0.3329 | 0.4165 | 0.6809 | 0.0605 | |
| Smoker | 0.3423 | 0.6184 | 0.5685 | 0.2596 | 0.4939 | 0.4668 |
| Diabetes | 0.7453 | 0.2459 | | 0.9382 | 0.5891 | 0.6332 |
| LAVI | 0.0619 | 0.0351 | 0.0307 | 0.0395 | 0.0121 | |
| LVVI | 0.0069 | −0.0069 | 0.0309 | 0.0146 | 0.0031 | 0.0184 |
| RAVI | 0.0204 | −0.0069 | | 0.0052 | −0.0151 | |
| RVVI | −0.0256 | −0.0418 | −0.0446 | −0.0058 | −0.0121 | |
| LVWI | 0.0185 | 0.0425 | | 0.0238 | 0.0313 | |
| Ln(Agatston + 1) | 0.3243 | 0.1924 | 0.2007 | 0.3045 | 0.2337 | 0.2431 |
| Baseline survival | | 0.9565 | 0.9556 | | 0.9192 | 0.9242 |
| Mean (var) × coef | | 2.6693 | 1.6915 | | 4.5851 | 5.1488 |

In certain embodiments, system 200 can include display 220 configured for displaying, for example, cardiovascular structures, estimated volumes, and/or a graphic representation of the risks determined by risk calculator 215. In one embodiment, computer enabled display 220 can be on a mobile application or a web application that can be used by patients and/or care providers. In some embodiments, computer enabled display 220 can be on a desktop application run on premises to, for example, avoid patient data security concerns.

CARDIAC EJECTION FRACTION MEASUREMENT USING NON-CONTRAST CT SCANS. Cardiac ejection fraction also referred to as left ventricular ejection fraction (EF) is a measurement of the amount of blood pumped out of the heart into the body through the aorta with each heartbeat. EF is typically expressed as a percentage, and it can be an important indicator of heart health. A common method of measuring EF uses echocardiography, which uses ultrasound waves to create images of the heart. The images can be used to measure the blood volume in the heart before a heartbeat (EDV) and after a heartbeat (ESV). EF is calculated by dividing the blood volume pumped out of the heart (EDV-ESV) by EDV.

Other methods for measuring EF use magnetic resonance imaging (MRI) and computed tomography (CT) scans. These imaging techniques can provide more detailed images of the heart, but they are more expensive and may not be readily available in all healthcare settings. Gated-ECG, contrast-enhanced CT scans can be used to measure EF, and such scans can be useful in certain clinical situations where other methods are not feasible.

Contrast agents are substances that are injected into the body to make tissues or structures more visible for imaging. However, injecting a patient with a contrast agent can damage, for example, the kidneys. Contrast-induced nephropathy (CIN) is a type of kidney damage that can occur after a patient receives a contrast agent for medical imaging procedures, such as CT scans, angiograms, or X-rays. In severe cases, CIN can lead to acute kidney injury or even kidney failure, requiring dialysis or other forms of treatment. The risk of developing CIN depends on several factors, including the type and dose of contrast agent used, the patient's age, underlying medical conditions such as kidney disease or diabetes, and the presence of other risk factors such as dehydration or the use of certain medications.

During CT scans, a patient's heart rate and rhythm can be monitored using an electrocardiogram (ECG). ECG is a non-invasive medical test that records the electrical activity of the heart over a period of time. The ECG signals can be used to synchronize the CT scanner with the patient's heartbeat, allowing acquisition of heart images at specific points in the cardiac cycle.

Retrospective ECG gating and prospective ECG gating are two methods used in CT imaging to reduce the effect of motion artifacts from the beating heart during image acquisition. The main difference between these two methods is the timing of image acquisition during the cardiac cycle.

Retrospective ECG gating involves acquiring ECG data and images continuously and simultaneously throughout the cardiac cycle. This method uses a computer to sort the ECG data into separate phases of the cardiac cycle, typically using the R-wave of the ECG signal as a reference point. The images acquired are then sorted into different phases of the cardiac cycle, and reconstructed into a final image that represents a single phase of the cycle. Retrospective ECG gating provides high temporal resolution and allows for the reconstruction of images at any point in the cardiac cycle. However, compared to prospective ECG gating, retrospective ECG gating exposes the patient to a much higher level of X-ray radiation.

Retrospective ECG gating is generally used to measure cardiac function. Because images are acquired throughout the cardiac cycle, volume measurements of the right and left ventricles can be obtained in the end-systole phase (ES) and end-diastole phase (ED), allowing the calculation of stroke volume, EF, and cardiac output. Retrospective ECG gating can also facilitate acquiring diagnostic images of the coronary arteries. In contrast to prospective ECG gating, retrospective ECG gating facilitates ECG editing to remove artifacts related to premature ventricular contractions and/or dropped beats.

Prospective ECG gating involves acquiring images only during a specific phase of the cardiac cycle, typically during diastole when the heart is at rest. The ECG signal is used to trigger image acquisition only during this phase of the cycle. This method provides lower temporal resolution than retrospective ECG gating, but the advantage is that it reduces radiation exposure.

An AI-enabled technique can be used for automated cardiac chambers volumetry (for example, AutoChamber) in non-contrast CT scans (for example, chest CT scans). The technique estimates cardiac chamber volume (including, for example, LV volume) using non-contrast, gated and/or non-gated CT scans. This technique can detect individuals (even asymptomatic individuals) at high risk of adverse health conditions such as heart failure, atrial fibrillation, and stroke.

In one embodiment, the invention is directed to a method of measuring EF. The method can include acquiring ECG-gated CT scans during ED (usually around 75%) and at ES during “isovolumic relaxation” which is around 25%. In some embodiments, using AutoChamber, the change in LV volume is measured to estimate EF. The change in the entire LV and LV wall can also be measured to estimate EF. Compared to retrospective ECG-gating, which requires contrast-enhanced CT scans, this AI-enabled technique reduces X-ray radiation significantly and does not require injecting patients with contrast agents.

In certain embodiments, this technique can be used in addition to screening with AutoChamber. For example, if there is an indication of an enlarged chamber, then a second scan can be done to measure EF. The second scan can be done immediately after the first scan and can be limited to ES only, or it can be a full-set of ED plus ES snapshot images. In the former scenario, AutoChamber can be embedded in the CT scan to facilitate an immediate decision on whether to obtain an EF measurement.

In one embodiment, the accuracy of the EF measurement can be improved by acquiring images during the “isovolumic contraction” phase and the “isovolumic relaxation” phase. During isovolumic contraction and isovolumic relaxation the volume is, respectively, maximum and minimum. In some embodiments, this method can be used as an embedded, AI-enabled algorithm of any ECG-gated, CT scan used for cardiac imaging.

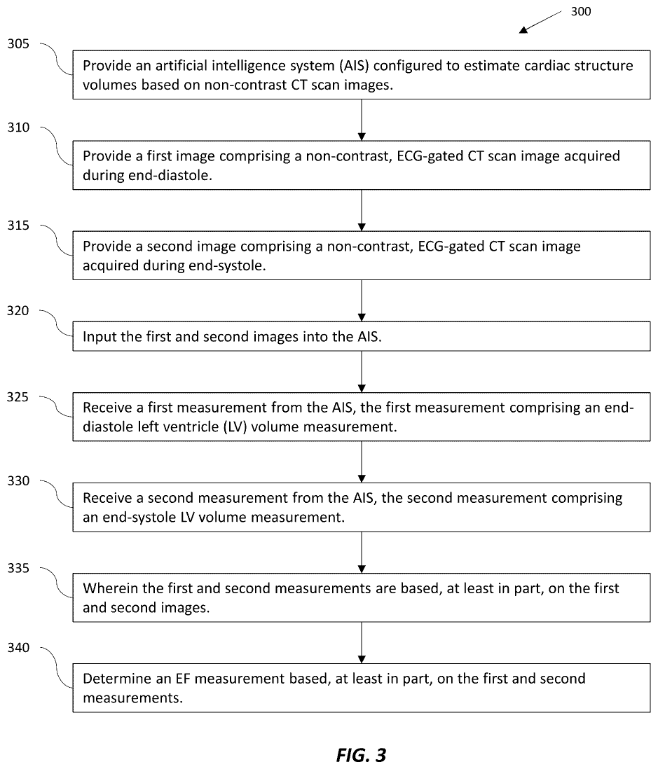

FIG. 3 illustrates AI-based method 300 of measuring cardiac ejection fraction (EF) according to one embodiment of the invention. At step 305, an artificial intelligence system (AIS) can be provided; the AIS is preferably configured to estimate cardiac structure volumes based on non-contrast CT scan images. At step 310, it is provided an image that can be a non-contrast, ECG-gated CT scan image acquired during end-diastole (“end-diastole image” or “first image”). At step 315, it is provided another image that can be a non-contrast, ECG-gated CT scan image acquired during end-systole (“end-systole image” or “second image”). At step 320, the first and second images are input into the AIS.

At steps 325 and 330, measurements for end-diastole left ventricle (LV) volume (“first measurement”) and end-systole LV volume (“second measurement”) are received. In one embodiment, said measurements are based, at least in part, on the end-diastole and end-systole images (335). At step 340, an EF measurement is determined based, at least in part, on the first and second measurements.

In one embodiment, the first and second images are acquired during a prospective ECG-gated CT scan. In some embodiments, the first image is acquired during isovolumic contraction, and the second image is acquired during isovolumic relaxation. In certain embodiments, a measurement of LV wall volume can be received from the AIS.

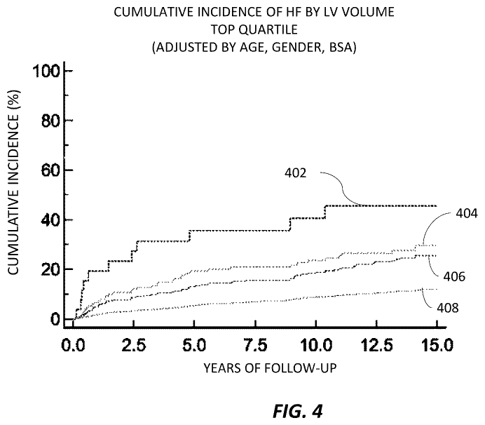

FIG. 4 shows cumulative incidence rates of HF using LV volume estimates performed by embodiments of system 200 using non-contrast CAC scans. FIG. 4 shows cumulative incidence rates of HF among certain top percentiles of LV volume estimates, as adjusted by age, gender, and body surface area. It is shown top 1% 402, top 5% 404, top 10% 406, and top 25% 408. Risk calculator 215 can be configured to automatically determine a risk of HF by using the LV volume estimates produced by AI-based analyzer 205.

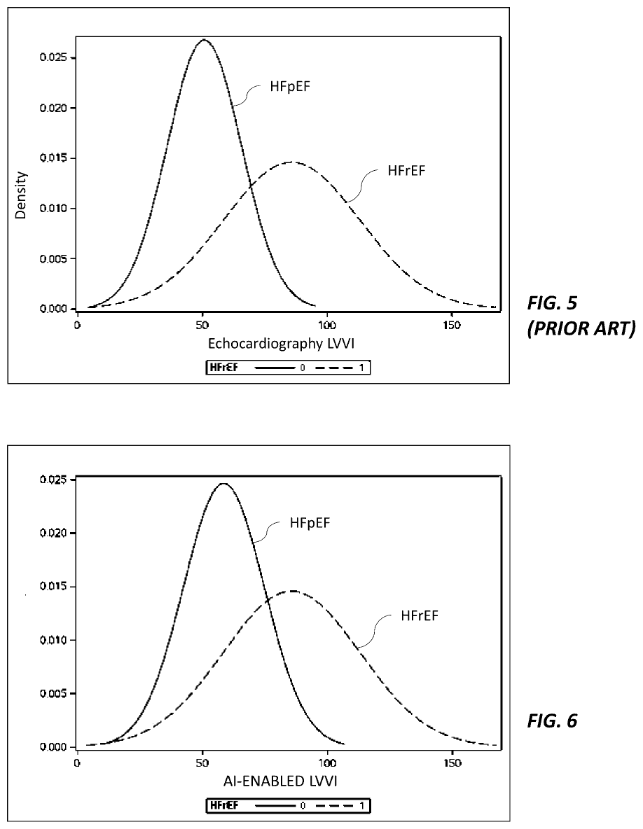

Heart failure with preserved ejection fraction (HFpEF) is typically defined as heart failure with a left ventricular ejection fraction (LVEF) of 50% or greater. Heart failure with a reduced ejection fraction (HFrEF) is heart failure with an LVEF of 40% or less. FIG. 5 shows HFrEF versus HFpEF by LV volume as performed by certain embodiments of the inventive systems and methods disclosed herein. FIG. 6 shows HFrEF versus HFpEF by left ventricle volume index (LVVI) as performed by certain embodiments of the inventive systems and methods disclosed herein. LVVI is defined as LV volume divided adjusted by body surface area. Some embodiments of the inventive systems and methods disclosed herein can facilitate distinguishing whether a patient is associated with HFpEF or, rather, HFrEF. Being able to make such a distinction can facilitate selecting the most adequate healthcare intervention that applies to the patient.

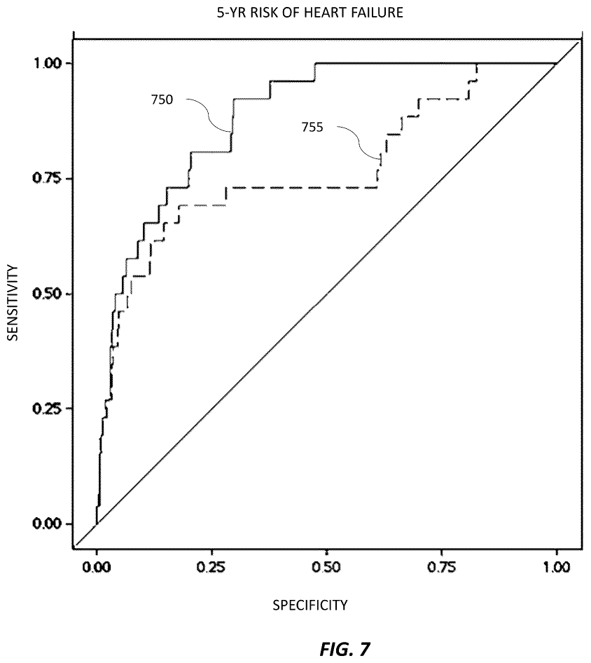

FIG. 7 illustrates the prediction performance of an AI-enabled model, according to the inventive systems and methods disclosed herein, versus the prediction performance of BNP, a blood test used for heart failure studies. As shown, the AI-enabled prediction model 750 significantly outperforms BNP prediction model 755. The ROC Curve (Area) for AI-enabled model 750 is 0.8849 and for BNP model 755 is 0.7660, in this example. Risk calculator 215 can be configured to automatically determine a risk of HF by using EF produced by AI-based analyzer 205.

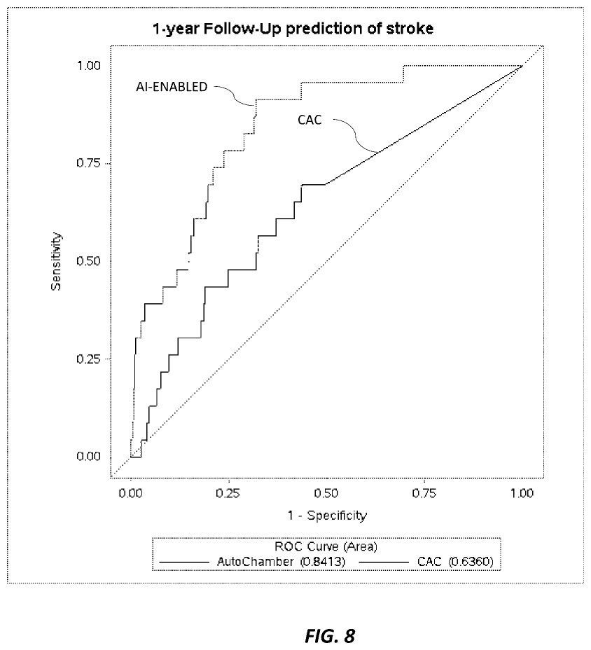

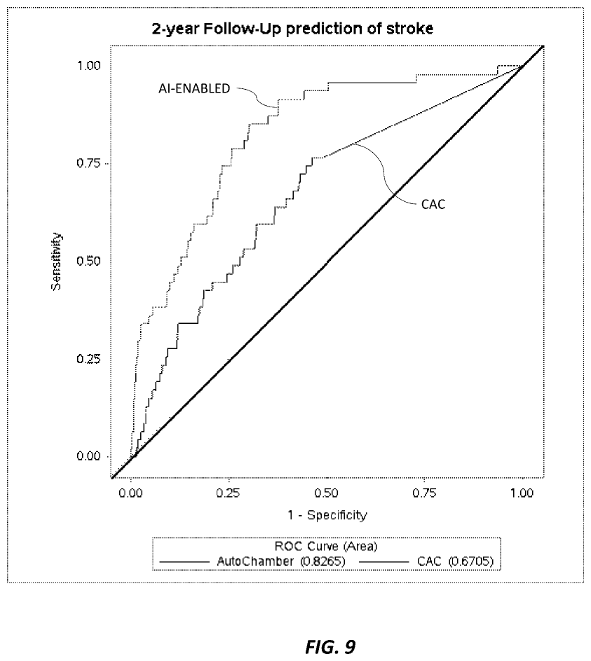

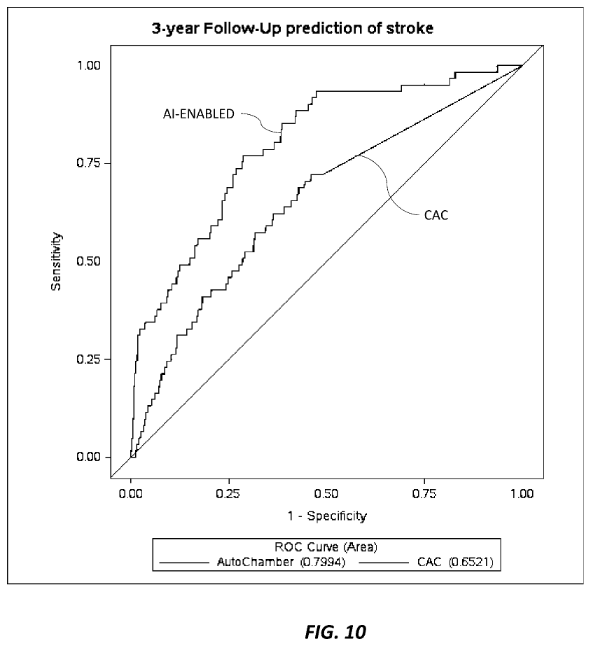

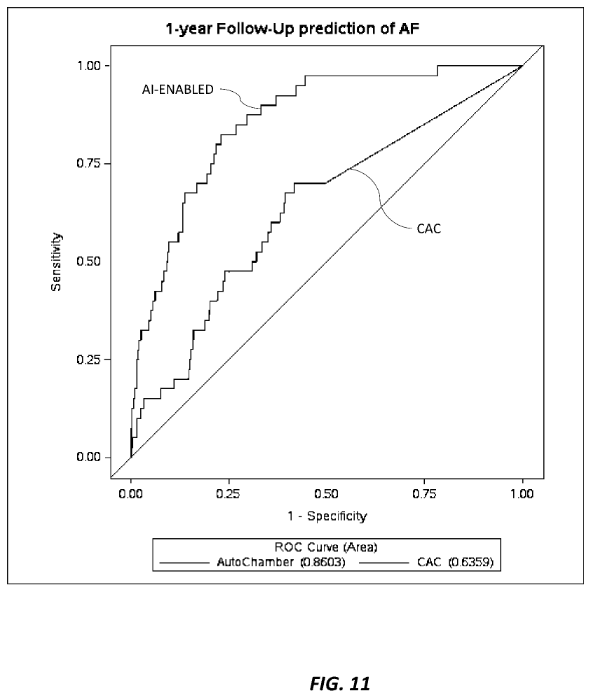

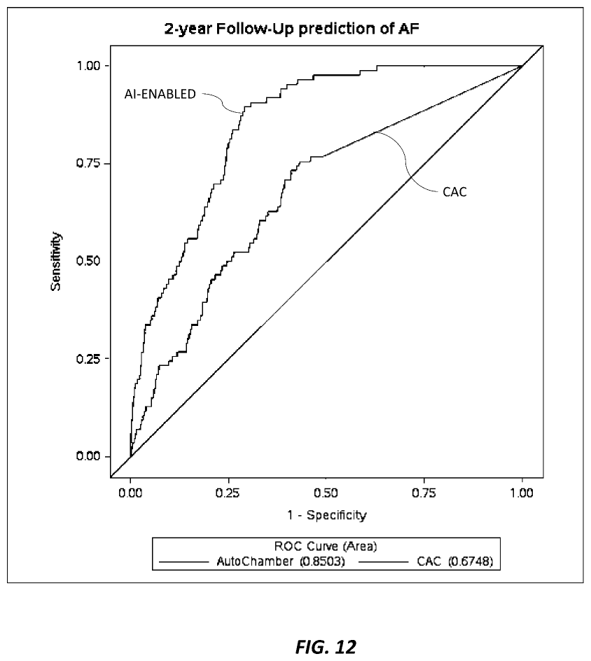

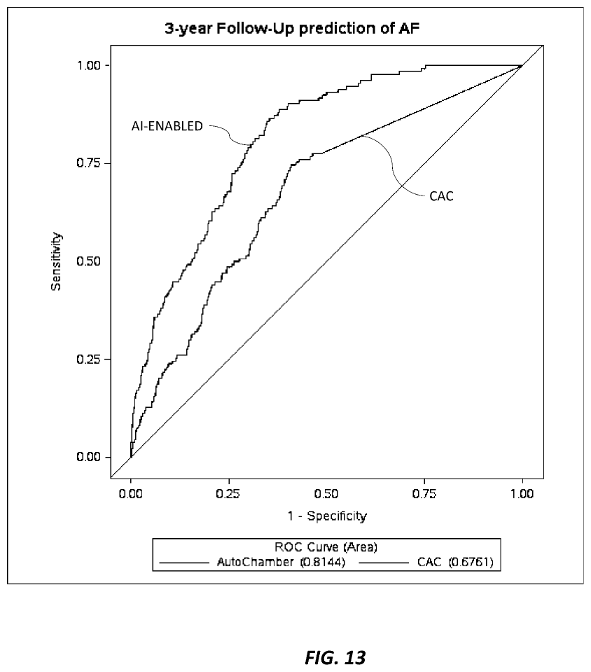

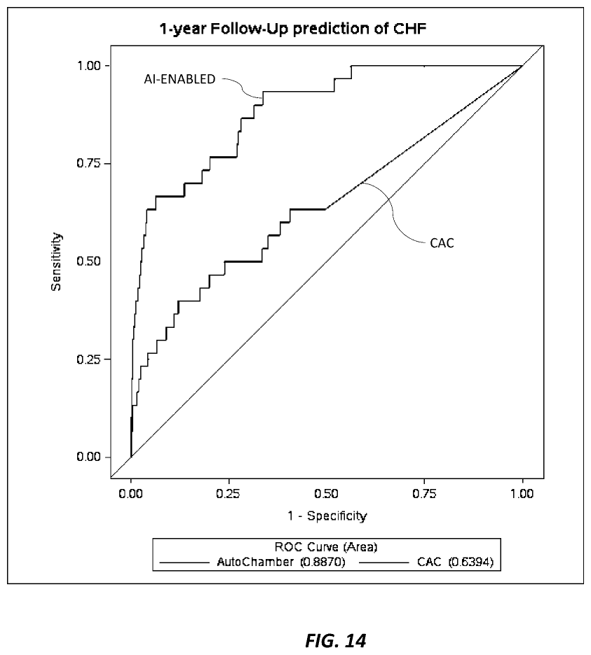

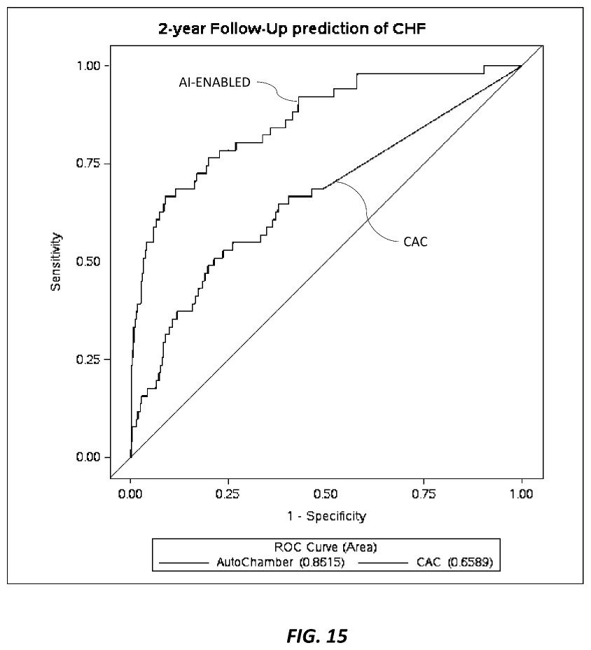

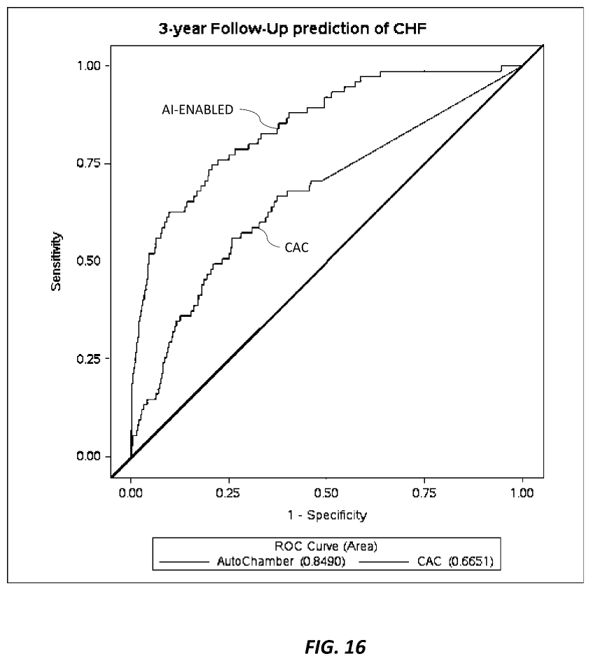

FIG. 8-10 illustrate risk assessment for stroke using embodiments of systems and methods disclosed here. Respectively, FIG. 8, FIG. 9, and FIG. 10 show prediction of stroke within 1-year, 2-years, and 3-years. FIG. 11-13 illustrate risk assessment for atrial fibrillation (AF) using embodiments of systems and methods disclosed here. Respectively, FIG. 11, FIG. 12, and FIG. 13 show prediction of AF within 1-year, 2-years, and 3-years. FIG. 14-16 illustrate risk assessment for congestive heart failure (CHF) using embodiments of systems and methods disclosed here. Respectively, FIG. 14, FIG. 15, and FIG. 16 show prediction of CHF within 1-year, 2-years, and 3-years.

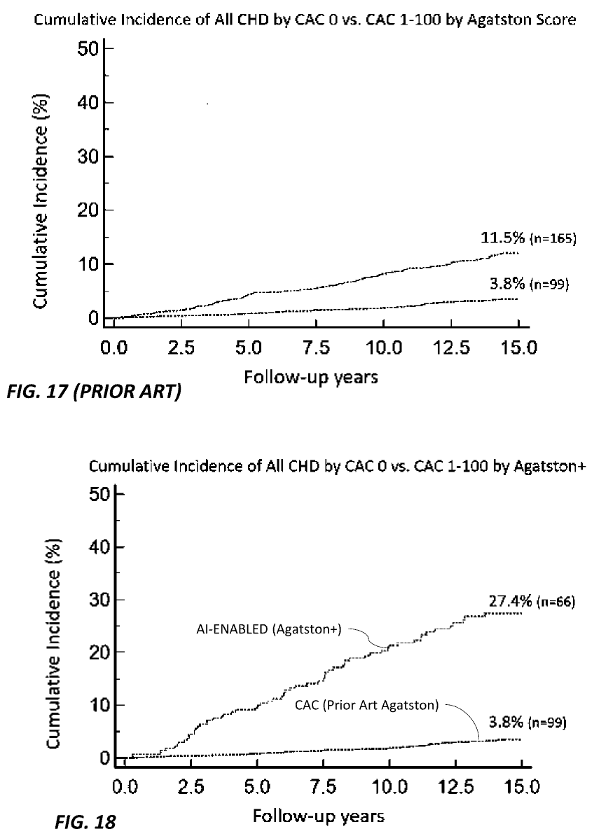

FIG. 17 illustrates cumulative incidence of all coronary heart disease (CHD) based on a prior art Agatston CAC score of zero and a prior art Agatston CAC score of 1-100. FIG. 18 shows cumulative incidence of all CHD based on a prior art Agatston score of zero as compared with an AI-enabled, modified Agatston score (“Agatston+”) based on the systems and methods disclosed here.

The table below shows the improvements of Agatston+ over prior art Agatston CAC score in prediction of various adverse health outcomes at 5-year, 10-year, and 15-year follow up periods. This table shows the net reclassification index (NRI) of cardiac chambers volumetry when added to the Agatston CAC score. Any NRI over 0.1 is viewed as clinically meaningful and NRI over 50 is viewed as very effective.

| NRI Improvement over CAC | 5-YR | 10-YR | 15-YR |
|---|---|---|---|
| AF | 0.56 | 0.43 | 0.33 |
| CHF | 0.78 | 0.63 | 0.37 |
| CHD HARD | 0.01 | 0.01 | 0.02 |
| CHD ALL | 0.06 | 0.02 | 0.01 |
| STROKE | 0.88 | 0.88 | 0.38 |
| CVD HARD | 0.57 | 0.40 | 0.44 |
| CVD ALL | 0.49 | 0.41 | 0.41 |
| ALL CAUSE MORTALITY | 0.64 | 0.63 | 0.57 |

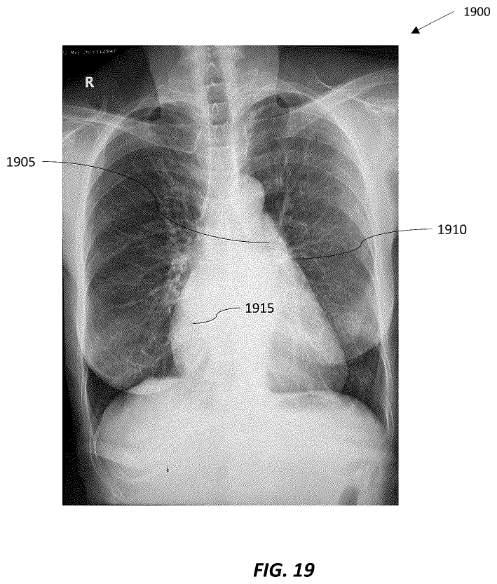

FIG. 19 illustrates a 2-D x-ray image 1900 analyzed (by AI-based analyzer 205 (FIG. 2), for example) to determine certain features that can be used to produce a modified Agatston score. In image 1900 prominent main pulmonary artery 1905, enlarged left atrial appendage 1910, and shadow in shadow appearance of left atrial enlargement 1915 have been identified.

In certain embodiments, a modified Agatston Score can be produced by taking into account one or more of the following: 1) the location of plaque (whether the plaque is in the beginning, middle, or end of a coronary artery); 2) the number of coronary arteries that have a plaque; 3) the mean density of HU units in a plaque (in the prior art Agatston score only the maximum intensity is counted); 4) the standard deviation of HU per plaque; and 5) the distance between two plaques or multiple plaques. In some embodiments, if there is low or no calcium, the process is performed again with HU 100-130 (which is 30 units below the prior art Agatston score cut-off point) to look for tiny amount of calcium. Using an AIS trained on background noises in non-contrast CT scan and on 3D coronary artery zones, this method can facilitate detecting coronary arteries with non-calcified (that is, soft plaques) and raise flags for further medical attention including a recommendation of contrast enhanced coronary CT angiography (CCTA) to provide delineation of coronary artery wall.

In certain embodiments of this invention, and referencing here U.S. provisional application 63/414,561, obtaining a CCTA after a non-contrast CT can facilitate identifying plaques with excessive vasa vasorum (angiogenesis) surrounding the plaque that is active or inflamed. The more vasa vasorum the more blood flow and contrast agent surrounding the plaque therefore showing higher HU. An active plaque is usually less calcified than a passive or stable plaque. By detecting passive plaques with CAC score and detecting active plaques with vasa vasorum density, embodiments of this invention can cover significant aspects of coronary risk assessment.

One motivation for the inventive embodiments described herein is the need for systems and methods to facilitate a more accurate prediction of adverse health outcomes, as compared to the current state of the art. Embodiments of the invention, for example with a combination of cardiac chambers volumetry and a coronary calcium score, can provide an unprecedented ROC AUC of 0.90 for prediction of stroke in 12 months. With the prior art Agatston score, about 3% of individuals with Agatston Score CAC=0 in a 15-yr follow up study had a heart attack. With the modified Agatston score (“Agatston+”) produced by the systems and methods disclosed here, it can facilitate assigning a more accurate and reliable CAC score of zero.

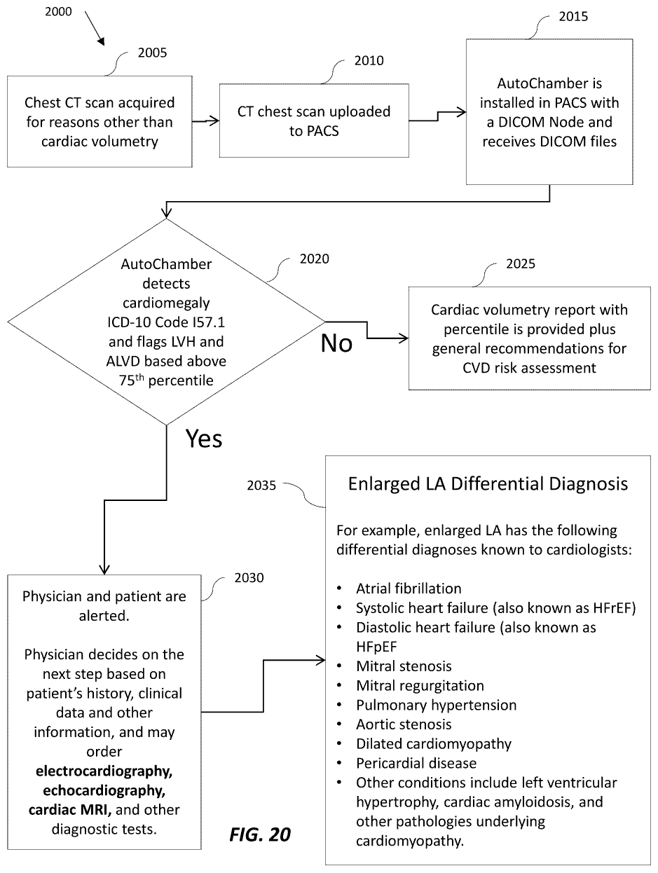

FIG. 20 illustrates one embodiment, according to the invention, of a method of determining a risk of an adverse health outcome. Method 2000 can be a workflow that, in some embodiments, can be based on a CACplus AutoChamber workflow provided by HeartLung Corporation. At step 2005 a chest CT scan is acquired for reasons other than cardiac volumetry. At a step 2010, the CT chest scan is uploaded to PACS. At a step 2015, AutoChamber can be installed in PACS with a DICOM Node and receives DICOM files. At a step 2020, AutoChamber detects whether there is a cardiomegaly ICD-10 Code 157.1 and flags cases high risk for LVH (Left Ventricular Hypertrophy) and ALVD (Asymptomatic Left Ventricular Dysfunction). At a step 2025, if AutoChamber does not detect ICD-10 Code 157.1, a cardiac volumetry report with percentile is provided plus general recommendations for CVD risk assessment. At a step 2030, if AutoChamber detects ICD-10 Code 157.1, a physician and a patient are alerted. The physician decides on the next step based on the patient's history, clinical data and other information, and may order electrocardiography, echocardiography, cardiac MRI, and other diagnostic tests. Enlarged LA Differential Diagnosis. For example, enlarged LA has the following differential diagnoses known to cardiologists: Atrial fibrillation, Systolic heart failure (also known as HFrEF), Diastolic heart failure (also known as HFpEF, Mitral stenosis, Mitral regurgitation, Pulmonary hypertension, Aortic stenosis, Dilated cardiomyopathy, Pericardial disease, other conditions include left ventricular hypertrophy, cardiac amyloidosis, and other pathologies underlying cardiomyopathy.

1. An AI-based method of assessing the risk of adverse health outcomes, the method comprising:
providing a first artificial intelligence system (AIS1) configured to analyze a set of computed tomography (CT) scan images;
inputting a set of CT scan images into AIS1 for analysis;
receiving an AI analysis from AIS1, the AI analysis comprising:
a coronary artery calcium (CAC) score based, at least in part, on an Agatston score;
a measurement of mean, median, minimum, maximum, and standard deviation of Hounsfield units (HU) per plaque;
a measurement of plaque quantity per coronary artery and per patient;
a measurement of the number of coronary arteries with one or more plaque;
a measurement of location of plaques from proximal to the aortic root, to distal parts of a coronary artery;
a measurement of left atrial volume;
a measurement of left ventricular volume;
a measurement of right atrial volume;
a measurement of right ventricular volume;
a measurement of left ventricular wall mass;
wherein the AI analysis is based at least in part on the set of CT scan images;
providing a second AIS (AIS2) configured to classify certain data associated with coronary plaques and cardiac chambers based on the output of AIS1; and
determining, based at least in part on a computerized risk calculator, the AIS1 analysis, the classification performed by AIS2, and known risk factors, a patient's risk for an adverse health outcome.

2. The method of claim 1, wherein the known risk factors comprise one or more of age, gender, ethnicity, smoking, abnormal blood pressure, blood lipids, blood glucose, electrocardiogram, hemoglobin A1C, brain natriuretic peptide, presence of diabetes, family history of heart disease, presence of vascular dysfunction and cardiometabolic syndromes.

3. The method of claim 1, wherein the AI analysis further comprises a measurement of at least one of: plaque shape, aortic valve calcification, aortic wall calcification, aortic diameter, pulmonary arteries diameter, lung parenchyma density, emphysema score, lung nodules, lung airways, thyroid nodules, thoracic lymph nodes, thymus, esophagus, hiatal hernia, thoracic bone mineral density, scoliosis, kyphosis, pericardial fat, intra thoracic fat, liver fat, subcutaneous fat, and thoracic muscle mass.

4. The method of claim 3, wherein determining, based at least in part on a computerized multi-variate risk calculator, a patient's risk for an adverse health outcome further comprises configuring the risk calculator for specialized assessment of different adverse outcomes selected from the group comprising: coronary heart disease, congestive heart failure, atrial fibrillation, left ventricular hypertrophy, hypertrophic obstructive cardiomyopathy, stroke, chronic obstructive pulmonary disease, lung cancer, thyroid cancer, metastatic cancer, non-alcoholic fatty liver disease, cardiovascular death, and all-cause mortality.

5. The method of claim 4, wherein determining, based at least in part on a computerized multi-variate risk calculator or an AI alert system to warn patients of an imminent risk of an adverse event. The risk forecaster is further configured to provide an alert of an imminent risk comprises days, weeks, and/or up to 12 months.

6. The method of claim 1, wherein the AI analysis further comprises a measurement of plaque calcifications defined by HU≥100.

7. The method of claim 1, wherein the set of CT scan images comprises images obtained from contrast-enhanced cardiac CT scans.

8. The method of claim 3, further comprising monitoring the amount of calcification in the coronary arteries, cardiac valves, aortic valve, aortic wall calcification, aortic diameter, pulmonary arteries diameter, lung parenchyma density, emphysema score, lung nodules, lung airways, thyroid nodules, thoracic lymph nodes, thoracic bone mineral density, scoliosis, kyphosis, pericardial fat, intra thoracic fat, liver fat, subcutaneous fat, and thoracic muscle mass, over time to evaluate progression, regression, or measuring response to therapies.

9. The method of claim 7, wherein the AI analysis further comprises a display of vasa vasorum density (also known as angiogenesis) around coronary arteries by mapping the Hounsfield units where the areas corresponding to highest vasa vasorum density have the highest Hounsfield units hinting an active inflammatory area.

10. The method of claim 2, wherein determining a patient's risk for an adverse health outcome is further based, at least in part, on an emerging biomarker selected from polygenic risk score and physiological testing data from electrocardiography, echocardiography, cardiac MRI, and/or photoplethysmography.

11. An AI-based system for facilitating risk assessment of adverse health outcomes, the system comprising:
a device configured to facilitate acquiring and storing a set of CT scan images;
an AI-enabled analyzer configured to generate an analysis based, at least in part, on said set of scan images;
wherein the analysis comprises:
a coronary artery calcium (CAC) score based, at least in part, on an Agatston score;
a measurement of mean, median, minimum, maximum, and standard deviation of Hounsfield units (HU) per plaque;
a measurement of plaque quantity per coronary artery and per patient;
a measurement of the number of coronary arteries with one or more plaque;
a measurement of location of plaques from proximal to the aortic root, to distal parts of a coronary artery;
a measurement of left atrial volume;
a measurement of left ventricular volume;
a measurement of right atrial volume;
a measurement of right ventricular volume;
a measurement of left ventricular wall mass; and
a computer-enabled risk calculator configured to determine, based at least in part on the analysis, a particular patient's risk for an adverse health outcome.

12. The system of claim 11, wherein the system is configured as a mobile CT scan unit to facilitate rapid screening services for early detection of heart disease in asymptomatic individuals.

13. The system of claim 11, wherein the particular patient's risk is electronically transferred to, stored on and displayed via an app executed on a computing device.

14. An AI-enabled method of measuring cardiac ejection fraction (EF), the method comprising:
providing an artificial intelligence system (AIS) configured to estimate cardiac chambers volumes based on non-contrast ECG-gated CT scan images of the heart;
providing a first image comprising a non-contrast, ECG-gated CT scan image of the heart acquired during end-diastolic period;
providing a second image comprising a non-contrast, ECG-gated CT scan image of the heart acquired during end-systolic period;
wherein the first and second measurements are based, at least in part, on the first and second images;
calculating the difference in left ventricular volume between the first and second images; and
determining an EF measurement based, at least in part, on the first and second measurements.

15. The method of claim 14, wherein the first image is acquired during isovolumetric contraction, and wherein the second image is acquired during isovolumetric relaxation.

16. The method of claim 14, further comprising receiving from the AIS a third measurement comprising a measurement of LV wall volume and total heart volume to calculate total heart volume changes between end-diastolic period and end-systolic period.

17. An AI-enabled system (AIS) configured to facilitate detecting individuals at high risk of future adverse health outcomes, the system comprising:
an AI-based module configured to receive and analyze 2-D chest X-ray images; and
a computerized calculator configured to detect individuals at high risk of one or more of the following: atrial fibrillation, heart failure, and stroke;
wherein the AI-based module is configured to perform an analysis based, at least in part, on detecting in the X-ray images characteristics of an enlarged left atrium, enlarged left atrial appendage, enlarged right atrium, dilated pulmonary arteries, dilated pulmonary veins, and/or enlarged right and left ventricles, and increased density of lungs due to excess blood flow.

18. An AI-enabled system (AIS) configured to improve on the clinical utility of CAC scans, the system comprising:
an AI-based module configured to extract from the CT scans a CAC scans cardiac score, cardiac chambers volumetry data, and thoracic vertebral bone mineral density data;
a computerized CVD risk calculator configured to provide a minimum Net Reclassification Index of 0.1 for risk assessment of individuals at risk of future heart failure, atrial fibrillation, stroke, LVH, ALVD, CVD related death and all-cause mortality; and
wherein the computerized CVD risk calculator is configured to provide the Net Reclassification Index based, at least in part, on the CAC scan cardiac score, the cardiac chambers volumetry data, and known risk factors from the group comprising: age, gender, smoking, and diabetes.
